(19) United States
(12) Patent Application Publication
(10) Pub. No.: US 2014/0311499 A1
(43) Pub. Date: Oct. 23, 2014

(54) HEADSET TO PROVIDE NOISE REDUCTION

(71) Applicant: SAMSUNG ELECTRONICS CO., LTD, SUWON-SI (KR)

(72) Inventors: Gun-woo LEE, Suwon-si (KR); Sang-chul KO, Seoul (KR); Young-sang LEE, Siheung-si (KR); Young-tae KIM, Seongnam-si (KR)

(73) Assignee: SAMSUNG ELECTRONICS CO., LTD, SUWON-SI (KR)

(21) Appl. No.: 14/256,251

(22) Filed: Apr. 18, 2014

(30) Foreign Application Priority Data
Apr. 19, 2013 (KR) 10-2013-0043726

(51) Int. Cl. H04R 3/00; G01R 33/28; A61F 11/14

(52) U.S. Cl. CPC H04R 3/002; A61F 11/14; G01R 33/288; A61F 2011/145
USPC 128/866; 381/71.6

(57) ABSTRACT

A headset to provide noise reduction may include a first microphone that is disposed outside the headset and detects external noise, a first blocking unit configured to block the external noise entering an inside of the headset, a second blocking unit configured to block external noise which is not blocked by the first blocking unit, a second microphone configured to detect internal noise of the headset including noise which is not blocked by the first blocking unit and the second blocking unit, and a speaker configured to output canceling noise to cancel the internal noise detected by the second microphone, wherein the second blocking unit surrounds the second microphone and comprises a one-way sound transmitting passage.

### CROSS-REFERENCE TO RELATED APPLICATIONS

This application claims the benefit under 35 U.S.C. §119(a) from Korean Patent Application No. 2013-0043726 filed Apr. 19, 2013 in the Korean Intellectual Property Office, the disclosure of which is incorporated herein by reference in its entirety.

### BACKGROUND OF THE INVENTION

1. Field of the Invention

The present disclosure relates to a headset, and more particularly, to a headset to provide noise reduction that can protect hearing by effectively blocking external noise by using an active noise control method and a passive noise control method.

2. Description of the Related Art

In the modern medical related field, importance of imaging equipment is increasing day by day. Hospitals are utilizing equipment such as an X-ray, computerized tomography (CT), a magnetic resonance imaging apparatus (MRI), etc., in order to diagnose and treat patients' illness more quickly and accurately. Various laboratories are utilizing equipment such as an f-MRI, etc., in order to provide studies on structure and function of the brain. Because the MRI has little effect on the human body and can obtain accurate images, it is a trend to use the MRI more and more among these various types of imaging equipment. However, there is a problem that noise being generated using the MRI in a process of obtaining an image of an affected part of a patient is too large. Accordingly, various techniques to solve noise from the MRI equipment have been invented.

Methods to reduce the noise reaching the patient in an MRI environment are largely divided into a passive control method and an active control method. The passive control method is a way to block noise from reaching the ear of a user by using a noise barrier. This method blocks the noise by using ear muffs or ear plugs. Alternatively, the method blocks a noise source itself by using materials to prevent vibration of the MRI equipment itself (a main cause of the MRI noise).

The active control method is a way to attenuate sound pressure by producing control signals that can cancel the MRI noise. However, a sound field control by an external speaker or a method to transmit sound generated by the external speaker to the inside of the headset through a tube, which was used as a conventional active control method, causes sound field disturbance and signal delay, thereby not providing practical noise control performance. Also, since a microphone is positioned at a distance from a human ear so as not to control actual noise inside the ear, the amount of noise to be canceled is limited while high-frequency noise cannot be canceled.

Also, although a headset type of speaker and microphone are used, locations of the speaker and microphone are varied depending on each of users wearing the headset or each time of wearing the headset by a user so that transfer paths between the speaker and the ear and between the microphone and the ear are unstable. Thus, overall noise reduction results can be minimal.

Accordingly, a noise control headset structure which has a passive noise isolation structure that does not depend on an individual's ear shape against external noise and can effectively cancel high levels of noise audible to the user by reflecting acoustic characteristics inside the ear to an algorithm is required.

### SUMMARY OF THE INVENTION

The present disclosure can overcome the above drawbacks and other problems associated with the conventional arrangement. The present disclosure provide a headset type of noise control apparatus that has a passive noise isolation structure that does not depend on an individual's ear shape in high noise levels of MRI environment and can cancel effectively high levels of noise audible to a patient by reflecting acoustic characteristics inside the ear to an algorithm, thereby protecting the auditory ear drum of the patient.

Additional features and utilities of the present general inventive concept will be set forth in part in the description which follows and, in part, will be obvious from the description, or may be learned by practice of the general inventive concept.

Exemplary embodiments of the present disclosure provide a headset for noise reduction, which may include a first microphone that is disposed outside the headset and detects external noise; a first blocking unit configured to block the external noise entering an inside of the headset; a second blocking unit configured to block noise, which is not blocked by the first blocking unit, of the external noise; a second microphone configured to detect internal noise of the headset including noise which is not blocked by the first blocking unit and the second blocking unit; and a speaker configured to output canceling noise for canceling the internal noise detected by the second microphone, wherein the second blocking unit surrounds the second microphone, and comprises a one-way sound transmitting passage.

The headset for noise reduction may include a cushion member which is connected to an edge of the first blocking unit, and in close contact with a human skin when wearing the headset, wherein the second blocking unit is connected to the cushion member inside the headset, and blocks noise, which is not blocked by the cushion member, of the external noise.

The second blocking unit may include a first transmitting passage through which the canceling noise outputted from the speaker moves; and a second transmitting passage through which final noise of the internal noise of the headset canceled by the canceling noise moves in a direction of a human ear.

The second blocking unit may include a close contact portion to allow the headset to be close to a human ear when wearing the headset, and may deliver final noise of the internal noise canceled by the canceling noise to a human ear through the sound transmitting passage.

The headset may include a headset for removing MRI noise.

The sound transmitting passage may be formed in a direction of an ear canal of a human ear when wearing the headset.

The first microphone may include an optical microphone, or an ECM microphone.

The second microphone may be disposed in the sound transmitting passage.

The speaker may output canceling noise for canceling internal noise detected by the second microphone based on characteristics information of the internal noise of the headset which is transmitted to an eardrum of a human.

The first blocking unit may include a partition wall configured to block the external noise entering the inside of the headset; and a sound absorbing unit which is disposed inside the partition wall, and absorbs noise entering the inside of the headset.

The first blocking unit may include a porous sound absorbing material.

The speaker may include a Piezo speaker.

The second blocking unit may include a close contact portion which allows the headset to be in close contact with a human ear when wearing the headset.

The headset for noise reduction may include a speaker sound emitting unit which provides a noise moving passage in a direction of a human ear so that noise generated in the speaker is canceled by the internal noise.

The speaker may be disposed parallel to a direction of the second microphone and the ear canal.

The cushion member may include a side surface to be connected to a lower case of the first blocking unit, a top surface exposed to an outside and other side surface to be in close contact with a skin of a human when wearing the headset.

According to various embodiments of the present disclosure, a headset type of noise control apparatus may include a passive noise isolation structure which does not depend on an individual's ear shape in high noise levels of MRI environment, and effectively cancels high-levels of noise audible to a patient by reflecting acoustic characteristics inside the ear to algorithm, thereby protecting an auditory organ of the patient

Exemplary embodiments of the present inventive concept may also include a headset providing noise reduction, comprising: a first microphone to detect external noise; an inner blocking unit disposed inside the headset and configured to block external noise; a second microphone configured to detect internal noise of the headset including noise which is not blocked by the blocking unit; and a speaker configured to output canceling noise to cancel the internal noise detected by the second microphone, wherein the blocking unit surrounds the second microphone and comprises a one-way sound transmitting passage.

In an exemplary embodiment, the blocking unit comprises: a first transmitting passage through which the canceling noise output from the speaker moves; and a second transmitting passage through which final noise of the internal noise of the headset canceled by the canceling noise moves in a direction toward a user's ear.

In an exemplary embodiment, the blocking unit comprises a close contact portion to allow the headset to be close to a user's ear when wearing the headset, and delivers final noise of the internal noise canceled by the canceling noise to a user's ear through the sound transmitting passage.

In an exemplary embodiment, the headset for noise reduction may further include a speaker sound emitting unit to provide a noise moving passage in a direction of a human ear so that noise generated in the speaker is canceled by the internal noise.

In an exemplary embodiment, the headset for noise reduction may further include a cushion member surrounding an edge of the headset to enclose the blocking unit and to come into close contact with a user to mitigate impact to the user and to block external noise before reaching the blocking unit.

### BRIEF DESCRIPTION OF THE DRAWINGS

These and/or other features and utilities of the present general inventive concept will become apparent and more readily appreciated from the following description of the embodiments, taken in conjunction with the accompanying drawings of which:

FIG. 1 is a sectional view illustrating a structure of a headset according to an embodiment of the present disclosure;

FIG. 2 is a schematic diagram illustrating a noise control method of a headset according to an embodiment of the present disclosure; and

FIG. 3 is a block diagram illustrating a noise control algorithm using transfer functions.

Throughout the drawings, like reference numerals will be understood to refer to like parts, components and structures.

### DETAILED DESCRIPTION OF THE PREFERRED EMBODIMENTS

Hereinafter, certain exemplary embodiments of the present disclosure will be described in detail with reference to the accompanying drawings.

The matters defined herein, such as a detailed construction and elements thereof, are provided to assist in a comprehensive understanding of this description. Thus, it is apparent that exemplary embodiments may be carried out without those defined matters. Also, well-known functions or constructions are omitted to provide a clear and concise description of exemplary embodiments. Further, dimensions of various elements in the accompanying drawings may be arbitrarily increased or decreased for assisting in a comprehensive understanding.

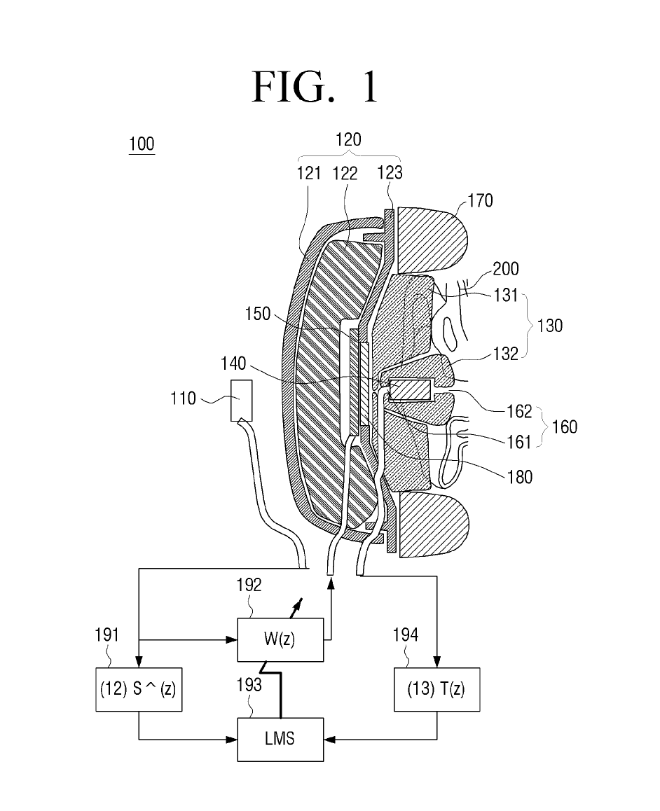

FIG. 1 is a sectional view illustrating a structure of a headset according to an embodiment of the present disclosure.

Referring to FIG. 1, a headset 100 according to an embodiment of the present disclosure includes a first microphone 110, a first blocking unit 120, a second blocking unit 130, a second microphone 140, a speaker 150, a sound transmitting passage 160, a cushion member 170, and a speaker sound emitting unit 180.

The first microphone 110 is placed outside the headset 100, and is configured to detect external noise. The first microphone 110 is a reference microphone to capture characteristics of the external noise for an active noise control. Because the first microphone 110 serves as a reference, it should be immune from being affected by surrounding electromagnetic fields. Accordingly, the first microphone 110 may be a microphone that can operate in the magnetic fields of the MRI such as an optical microphone, an electric capacitor microphone (ECM), etc.

The first blocking unit 120 is configured to block external noise from entering the inside of the headset 100. The first blocking unit 120 includes all configurations to block primarily the external noise.

First, the first blocking unit 120 includes cases 121 and 123 to enclose the inside of the headset 100. The cases 121 and 123 may be made up of reinforced plastic materials, and block MRI noise in a passive manner.

For the passive noise control, the first blocking unit 120 may include a sound absorbing material. In other words, as illustrated in FIG. 1, a sound absorbing material 122 is provided between the cases 121 and 123 to absorb noise passing through the cases 121 and 123. The sound absorbing material 122 may include porous materials such as rock wool, glass wool, texture, sponge, etc. If sound waves enter thin fibers and thin holes, vibration of air particles is converted into heat energy by frictional resistance of the inner surfaces of the thin holes and mutual friction of the fibers so that sound absorption takes place. The sound absorbing material 122 has a high sound absorbing rate in the high and mid frequency range of sound, but has a low sound absorbing rate in the low frequency range of sound. In order to increase the sound absorbing rate in the low-frequency range of sound, the thickness of the sound absorbing material 122 may be increased or an air layer may be installed in the rear portion of the headset. The passive noise control may effectively block the high-frequency noise, but may have a limitation with respect to completely blocking the low frequency noise, so there is a need to combine an active noise control with the passive noise control described above.

The second microphone 140 is disposed inside the headset 100, and collects noise within the inside of the headset 100. The second microphone 140 is used for the active noise control together with the first microphone 110. In detail, the external noise collected by the first microphone 110 and the internal noise collected by the second microphone 140 are compared with each other to determine output noise of the speaker 150, which will be described later. Because the second microphone 140 is also used for the active noise control so that accurate noise detection is important to the second microphone 140, the second microphone 140 should not receive a lot of influence from electromagnetic fields. Accordingly, the second microphone 140 may be a microphone which can operate in the MRI electromagnetic fields, such as an optical microphone, an ECM microphone, etc., while not being affected by these MRI electromagnetic fields.

The speaker 150 is configured to output canceling noise to remove the internal noise which is detected in the second microphone 140. In other words, the speaker 150 cancels the internal noise by generating noise having a phase opposite to the internal noise detected by the second microphone 140, thereby performing the active noise control. When being collected by the first microphone 110, the external noise is primarily blocked by the cases 121 and 123 of the first blocking unit 120, and then secondarily blocked by the sound absorbing material 122 inside the cases 121 and 123. However, noise remaining inside the headset 100 without being blocked is collected through the second microphone 140. Because the noise collected through the second microphone 140 eventually reaches and causes displeasure to the human ears, the speaker 150 generates and outputs noise having a frequency with a phase opposite to the noise collected in the second microphone 140. Because the speaker 150 is also used for the active noise control, it is important for the speaker 150 to output accurate noise. Accordingly, the speaker 150 should be configured as a speaker that is not affected by electromagnetic fields. For example, a speaker which can operate in the MRI electromagnetic fields, such as a Piezo speaker, may be used.

On the other hand, as illustrated in FIG. 1, the speaker 150 may be placed in a direction parallel to a direction in which the second microphone 140 and an ear canal of the human ear are placed. The noise may be accurately controlled by matching progress directions of the noise emitted from the speaker 150 and the noise collected by the second microphone 140, and the effect of the noise reduction may be delivered correctly to a human eardrum by matching the progress directions and the ear canal of the human.

The speaker sound emitting unit 180 provides a noise moving passage in a direction in which the human ear is located in order for the noise generated in the speaker 150 to be canceled with the internal noise.

The cushion member 170 is configured to be connected to the edge of the first blocking unit 120 and in close contact with the skin of a person wearing the headset 100. In more detailed, a side surface of the cushion member 170 is connected to the lower case 123 of the first blocking unit 120, a top surface thereof is exposed to the outside, and the other side surface (opposite to side connected to the lower case 123) thereof is close to the skin of the person wearing the headset 100. When wearing the headset 100, the cushion member 170 mitigates any impact to the human head, and is in close contact with the skin so that external noise is blocked. The cushion member 170 may use the same material as the sound absorbing material 122 as described previously. The cushion member 170 is connected to the second blocking unit 130 inside the headset 100, as is described in more detail later.

The second blocking unit 130 is configured to block external noise that may not be blocked by the first blocking unit 120 or the cushion member 170. As illustrated in FIG. 1, the second blocking unit 130 is connected to the cushion member 170 in the inside of the headset 100, and also blocks external noise which is not completely blocked by the cushion member 170.

Also, the second blocking unit 130 has a one-way or two-way sound transmitting passage 160 surrounding the second microphone 140 as described previously. In other words, the second blocking unit 130 blocks external noise including noise that was not blocked by the first blocking unit 120 or the cushion member 170 from moving in other directions, and allows the final noise to move through the sound transmitting passage 160.

The sound transmitting passage 160 is formed in the direction of the ear canal of the human ear, and includes a first transmitting passage 161 through which the canceling noise output from the speaker 150 moves and a second transmitting passage 162 to move the final noise that is the internal noise of the headset 100 canceled by the canceling noise in the direction of the human ear.

The first transmitting passage 161 receives the noise output from the speaker 150 and passed through the speaker sound emitting unit 180. Also, the internal noise collected by the second microphone 140 or the internal noise before being collected by the second microphone 140 is received by the first transmitting passage 161. As a result, the noise output from the speaker 150 and the internal noise meet in the first transmitting passage 161 to be canceled, and the canceled noise is collected in the second microphone 140. Then, the canceled noise moves to the ear canal through the second transmitting passage 162 connected to the second microphone 140.

The second transmitting passage 162 is connected to the first transmitting passage 161, and the second microphone 140 is placed in a space between the first transmitting passage 161 and the second transmitting passage 162. The noise which moves through the second transmitting passage 162 is noise that has been canceled by the active noise control, and is collected by the second microphone 140 after a certain point in time. Because the second blocking unit 130 forming the second transmitting passage 162 is in close contact with the human ear, the noise passing through the second transmitting passage 162 is directed to the ear canal of the human ear. As a result, the canceled noise is the noise that finally reaches the eardrum.

Also, the second blocking unit 130 includes a close contact portion 132 that allows the headset 100 to be in close contact with the human ear when wearing the headset 100. The close contact portion 132 is placed at an end of the second transmitting passage 162, and can be made of a soft elastic material. As a result, when wearing the headset 100, the headset 100 is firmly pressed against the user's ear regardless of the shape of the human ear. Also, the second blocking unit 130 secures the position of the second microphone 140 by surrounding the second microphone 140, thereby providing a fixed noise collecting environment and a uniform noise transmitting passage.

In addition, since the close contact portion 132 of the second blocking unit 130 is close to the user's ear, the second blocking unit 130 also has a function of providing passive noise control. In other words, the second blocking unit 130 blocks noise that would otherwise pass through a gap between the user's ear and the headset 100.

Hereinafter, operations of the headset 100 according to an embodiment of the present disclosure will be described.

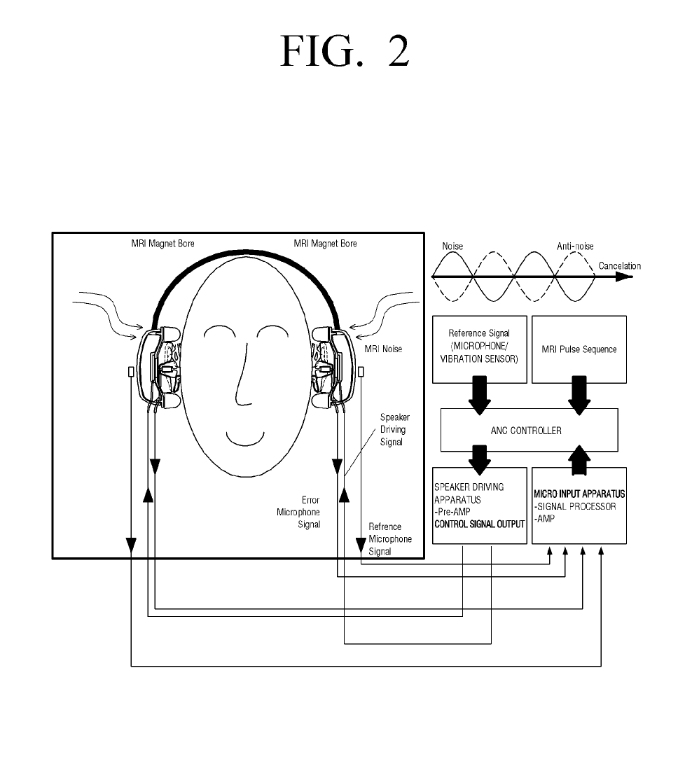

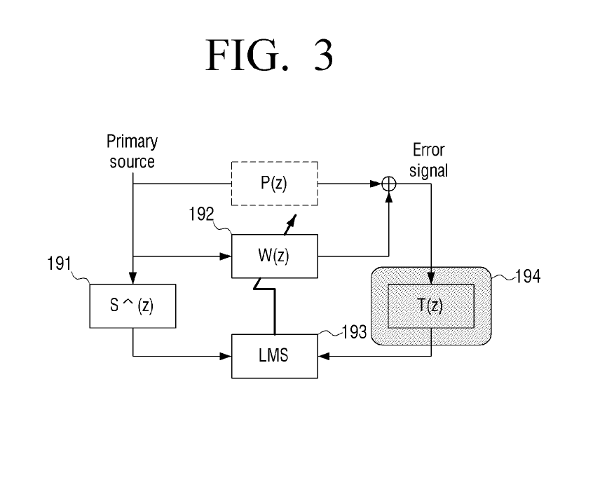

FIG. 2 is a schematic diagram illustrating a noise control method of a headset according to an embodiment of the present disclosure, and FIG. 3 is a block diagram illustrating noise control algorithm using transfer functions.

The headset 100 according to an embodiment of the present disclosure performs both passive noise control and active noise control. First, the passive noise control will be explained.

When wearing the headset 100 in an MRI environment, the cushion member 170 of the headset 100 is in close contact with the head of a user so as to passively block external noise. Then, the second blocking unit 130 (131 and 132) is in close contact with the user's ear so as to block noise that was not blocked by the cushion member 170. Likewise, the cases 121 and 123 of the first blocking unit 120 primarily block the external noise, and then the sound absorbing material 122 blocks the noise that may pass through the cases 121 and 123. The sound absorbing material 122 of the first blocking unit 120 and the second blocking unit 130 (131 and 132) passively block the high-frequency noise of the MRI noise. Also, the second blocking unit 130 (131 and 132) fills the internal space of the headset 100 so as to prevent the internal noise from being dispersed and to allow the noise to be canceled and moved through the noise passage. As a result, the second blocking unit 130 also plays an additional role of blocking low frequency noise.

At the same time, the headset 100 performs the active noise control. As illustrated in FIGS. 1 to 3, the external noise is primarily collected by the first microphone 110. When noise control algorithms operate during wearing the headset 100, a transfer function S(z) between the speaker 150 and the second microphone 140 may be measured or a pre-measured transfer function 191 may be used. Since the transfer function S(z) includes the characteristics of the sound absorbing material, it does not change significantly depending on the state of wearing the headset 100. Therefore, the transfer function S(z) can use a pre-measured value.

If the algorithm operates, the first microphone 110 receives the external noise, and then predicts in advance what characteristics of noise will reach the ear. Also, the second microphone 140 observes a change state of the sound pressure by measuring the internal noise near the user's ear.

The transfer function Ŝ (z) reflecting noise transfer characteristics of the first transmitting passage 161 is considered (S191) for the external noise detected in the first microphone 110. The transfer function Ŝ (z) reflects characteristics to be transformed in a process in which the noise output from the speaker 150 reaches the second microphone 140. The transfer function Ŝ (z) is sampled over a predetermined number of times, and is calculated statistically. A value of the transfer function Ŝ (z) is used as a parameter for setting speaker output noise in a least mean square error module (LMS) 193.

A transfer function T(z) is a transfer function between the second microphone 140 and the ear, and includes transfer function characteristics of the second blocking unit 130, a sound passage d made with it, and the ear canal, and may use statistical values by measuring these characteristics previously. By reflecting T(z), the algorithm may operate based on the sound pressure at the actual ear and not the sound pressure at the second blocking unit 130.

The transfer function T(z) is calculated as a difference therebetween by measuring characteristics T1(z) of the noise which the second microphone 140 collects and characteristics T2(z) of the second transmitting passage 162 of a transmitting passage 160 between the second microphone 140 and the ear. In other words, the transfer function T(z) may be calculated as follows (operation 194 illustrated in FIGS. 1 and 3)).

T(z)=T1(z)/T2(z)

The LMS 193 applies the transfer function T(z) against the noise detected in the second microphone 140, and calculates a filtering parameter to set output noise of the speaker 150. The transfer function T(z) (represented as t(n) in the following equation) is multiplied with the sound pressure e(n) of the second microphone 140 as a weight, like in the equation provided below. In the following equation, both the transfer function t(n) and the sound pressure e(n) of the second microphone 140 are defined as a function of time (n is a time variable).

n\hat{ξ}(n)=[e(n)*t(n)]²

A filter function W(z) generates canceling noise by using the external noise and the output value of the LMS 193. The speaker 150 outputs the generated canceling noise.

According to various embodiments of the present disclosure, the headset has a passive noise blocking structure that does not depend on the shape of individual's ears in the MRI environment, and effectively cancels high levels of noise audible to a user (i.e., an MRI patient) by making the algorithm reflect acoustic characteristics of the inside of the ear, thereby protecting a patient's auditory organ. Also, by the structure proposed in the present disclosure, the headset does not give discomfort to the user and can secure a distance as close as possible in a process in which the second microphone 140 approaches the user's ear, and can stably transmit the output of the speaker 150 to the ear or the second microphone 140, thereby obtaining a more efficient active noise control effect.

On the other hand, the noise control algorithm as described previously may be implemented as a program including an algorithm which can be executed in a computer, and the program may be stored in and provided with a non-transitory computer-readable medium.

Contrary to a medium to store data for a short moment, such as a register, a cache, a memory, etc., the non-transitory computer-readable medium refers to a medium that can store data in a semi-permanent manner and that can be read by devices. In detail, the above-described various applications or programs may be stored in and provided with the non-transitory computer readable medium such as a CD, a DVD, a hard disc, a Blu-ray disc, an USB, a memory card, a ROM, etc.

While the embodiments of the present disclosure have been described, additional variations and modifications of the embodiments may occur to those skilled in the art once they learn of the basic inventive concepts. Therefore, it is intended that the appended claims shall be construed to include both the above embodiments and all such variations and modifications that fall within the spirit and scope of the inventive concepts.

What is claimed is:

1. A headset to provide noise reduction, comprising:
a first microphone disposed outside the headset and detects external noise;
a first blocking unit configured to block the external noise entering an inside of the headset;
a second blocking unit configured to block external noise which is not blocked by the first blocking unit;
a second microphone configured to detect internal noise of the headset including noise which is not blocked by the first blocking unit and the second blocking unit; and
a speaker configured to output canceling noise to cancel the internal noise detected by the second microphone,
wherein the second blocking unit surrounds the second microphone and comprises a one-way sound transmitting passage.

2. The headset for noise reduction of claim 1, further comprising:
a cushion member connected to an edge of the first blocking unit and in close contact with a user when wearing the headset,
wherein the second blocking unit is connected to the cushion member inside the headset and blocks external noise which is not blocked by the cushion member.

3. The headset for noise reduction of claim 1, wherein the second blocking unit comprises:
a first transmitting passage through which the canceling noise output from the speaker moves; and
a second transmitting passage through which final noise of the internal noise of the headset canceled by the canceling noise moves in a direction toward a user's ear.

4. The headset for noise reduction of claim 1, wherein
the second blocking unit comprises a close contact portion to allow the headset to be close to a user's ear when wearing the headset, and delivers final noise of the internal noise canceled by the canceling noise to a user's ear through the sound transmitting passage.

5. The headset for noise reduction of claim 1, wherein the headset comprises a headset to remove MRI noise.

6. The headset for noise reduction of claim 1, wherein
the sound transmitting passage is formed in a direction of an ear canal of a user's ear when wearing the headset.

7. The headset for noise reduction of claim 1, wherein
the first microphone comprises an optical microphone or an ECM microphone.

8. The headset for noise reduction of claim 1, wherein
the second microphone is disposed in the sound transmitting passage.

9. The headset for noise reduction of claim 1, wherein
the speaker outputs canceling noise to cancel internal noise detected by the second microphone based on characteristics information of the internal noise of the headset which is transmitted to an eardrum of a user.

10. The headset for noise reduction of claim 1, wherein the first blocking unit comprises:
a partition wall configured to block the external noise entering the inside of the headset; and
a sound absorbing unit which is disposed inside the partition wall, and absorbs noise entering the inside of the headset.

11. The headset for noise reduction of claim 1, wherein
the first blocking unit comprises a porous sound absorbing material.

12. The headset for noise reduction of claim 1, wherein
the speaker comprises a Piezo speaker.

13. The headset for noise reduction of claim 1, wherein
the second blocking unit comprises a close contact portion which allows the headset to be in close contact with a human ear when wearing the headset.

14. The headset for noise reduction of claim 1, further comprising:
a speaker sound emitting unit which provides a noise moving passage in a direction of a human ear so that noise generated in the speaker is canceled by the internal noise.

15. The headset for noise reduction of claim 1, wherein
the speaker is disposed parallel to a direction of the second microphone and the ear canal.

16. The headset for noise reduction of claim 2, wherein the cushion member comprises:
a side surface to be connected to a lower case of the first blocking unit;
a top surface exposed to an outside; and
another side surface to be in close contact with a user when wearing the headset.

17. A headset providing noise reduction, comprising:
a first microphone to detect external noise;
an inner blocking unit disposed inside the headset and configured to block external noise;
a second microphone configured to detect internal noise of the headset including noise which is not blocked by the blocking unit; and
a speaker configured to output canceling noise to cancel the internal noise detected by the second microphone,
wherein the blocking unit surrounds the second microphone and comprises a one-way sound transmitting passage.

18. The headset for noise reduction of claim 17, wherein the blocking unit comprises:
a first transmitting passage through which the canceling noise output from the speaker moves; and
a second transmitting passage through which final noise of the internal noise of the headset canceled by the canceling noise moves in a direction toward a user's ear.

19. The headset for noise reduction of claim 17, wherein the blocking unit comprises a close contact portion to allow the headset to be close to a user's ear when wearing the headset, and delivers final noise of the internal noise canceled by the canceling noise to a user's ear through the sound transmitting passage.

20. The headset for noise reduction of claim 17, further comprising:
a speaker sound emitting unit to provide a noise moving passage in a direction of a human ear so that noise generated in the speaker is canceled by the internal noise.

21. The headset for noise reduction of claim 17, further comprising:
a cushion member surrounding an edge of the headset to enclose the blocking unit and to come into close contact with a user to mitigate impact to the user and to block external noise before reaching the blocking unit.
